(19) United States
(12) Patent Application Publication
(10) Pub. No.: US 2017/0252030 A1
(43) Pub. Date: Sep. 7, 2017

(54) ALL-SUTURE KNOTLESS REPAIR SYSTEM

(71) Applicants: Raymond Thal, McLean, VA (US); Jonathan Ticker, Glen Head, NY (US)

(72) Inventors: Raymond Thal, McLean, VA (US); Jonathan Ticker, Glen Head, NY (US)

(21) Appl. No.: 15/446,659

(22) Filed: Mar. 1, 2017

Related U.S. Application Data
(60) Provisional application No. 62/303,105, filed on Mar. 3, 2016.

(51) Int. Cl. A61B 17/04; A61B 17/06

(52) U.S. Cl. CPC A61B 17/0401; A61B 17/0487; A61B 17/06166; A61B 2017/0446; A61B 2017/0441; A61B 2017/0414; A61B 2017/0409

(57) ABSTRACT

An all-suture knotless repair system includes at least one all-suture anchor assembly, a tissue coupling suture assembly with a bone anchor, and a delivery inserter. The all-suture anchor assembly is composed of an anchor suture strand and a suture anchor. The suture anchor consists essentially of suture material.

### CROSS REFERENCE TO RELATED APPLICATION

This application claims the benefit of U.S. Provisional Patent Application Ser. No. 62/303,105, entitled “ALL-SUTURE KNOTLESS REPAIR SYSTEM,” filed Mar. 3, 2016.

### BACKGROUND OF THE INVENTION

### 1. Field of the Invention

The present invention relates to devices and/or methods used in tissue repair.

### 2. Description of the Related Art

Soft tissues, such as tendons and ligaments, generally are attached to bone by small collagenous fibers. These connections are strong but permit the tendons and ligaments to be flexible. When a tissue, or a portion of a tissue, is torn away from the bone and requires repair, a surgeon is often required to repair the detached soft tissue with sutures, which are passed through bone tunnels and tied. A number of devices have been developed for securing a ligament or tendon to a bone mass. These devices can be used in place of bone tunneling techniques. These attachment devices are usually applied through extensive surgical incisions and, in some circumstances, by arthroscopic surgical techniques. The placement of bone tunnels for repair can be difficult and generally requires large open incisions. Through the advent of arthroscopic surgery, where the surgeon looks into a joint cavity with an arthroscope, there has been a trend to repair soft tissues back to bone through small incisions called portals.

A variety of devices are available for attaching objects to bone, such as screws, staples, cement, suture anchors, and sutures alone. These devices have been used to attach soft tissue, such as ligaments, tendons, muscles, as well as objects such as prostheses, to bone. A suture anchor assembly is a device, which utilizes small anchors, including those made of suture material alone, with additional suture materials attached thereto. A device, such as a screw, is inserted into the bone mass and anchored in place. After insertion of the anchor device, the attached suture is passed through the tissue to be repaired. The tying of a knot in the suture is then required to secure the tissue to the bone. The process of passing the anchored suture through the soft tissue and tying a knot is time consuming and difficult to undertake in the tight space encountered during arthroscopic surgery and sometimes even in conventional open surgery.

Knotless anchor assemblies have been popular and are embodied in a number of prior patents such as U.S. Pat. No. 5,709,708 wherein there is provided an assembly with an anchor means having a snag means and a loop suture element attached thereto. The suture loop is passed through the tissue to be repaired. The snag means then captures the loop suture element. The anchor is then inserted into a bone hole in a bone mass and the anchor locks into the bone. As the anchor is inserted into the bone hole, the tissue is pulled into secure attachment with a bone mass.

Further, in U.S. Pat. No. 6,045,574 there is provided an assembly with an anchor means having a snag means, and a hollow sleeve element with a loop suture element attached thereto. The snag means captures a loop suture element of the hollow sleeve element to draw tissue into secure attachment with a bone mass.

Still further, there is provided an all-suture anchor assembly, such as disclosed in U.S. Patent Application Publication No. 2012/0290004 having an all fibrous construct, which is incorporated herein by reference. The device requires the tying of a knot to complete the surgical repair.

However, difficulties still exist with the all-suture anchor assembly and the present invention attempts to address these with a method and apparatus for knotless suture anchoring.

### SUMMARY OF THE INVENTION

It is, therefore, an object of the present invention to provide an all-suture knotless repair system including at least one all-suture anchor assembly, a tissue coupling suture assembly with a bone anchor, and a delivery inserter. The all-suture anchor assembly is composed of an anchor suture strand and a suture anchor. The suture anchor consists essentially of suture material.

It is also an object of the present invention to provide an all-suture knotless repair system wherein the anchor suture strand and the suture anchor are entangled in a manner facilitating bunching of the suture anchor causing compression of the suture anchor in a manner creating outward forces that allow for placement and anchoring of the suture anchor within a bone hole.

It is another object of the present invention to provide an all-suture knotless repair system wherein the suture material is an enlarged piece of cylindrical suture material or a suture tape.

It is further an object of the present invention to provide an all-suture knotless repair system wherein the suture anchor includes a first end and a second end, and the anchor suture strand is passed through the suture anchor at suture aperture locations along a length of the suture anchor so as to penetrate and traverse the suture anchor in a manner defining apertures in the suture anchor, intersections of the anchor suture strand with the suture anchor are the suture aperture locations.

It is another object of the present invention to provide an all-suture knotless repair system wherein the bone anchor includes an anchor body having threads for attachment of the anchor body to bone.

It is also an object of the present invention to provide an all-suture knotless repair system wherein a plurality of sutures extend from and are secured to the anchor body. Each of the sutures includes a first end secured to the anchor body and a second end that is selectively coupled with the all-suture anchor assembly such that the anchor body fixedly secures the first ends of the plurality of sutures at one location and the all-suture anchor assembly secures the second ends of the plurality of sutures at a second location.

It is another object of the present invention to provide an all-suture knotless repair system wherein the second ends of the plurality of sutures are secured to the all-suture anchor assembly by passing the second ends of the plurality of sutures between the anchor suture strand and the suture anchor at positions between the suture aperture locations such that when the all-suture anchor assembly is contracted to its compressed state friction is created between the second ends of the plurality of sutures and the all-suture anchor assembly in a manner securing the second ends of the plurality of sutures to the all-suture anchor assembly.

It is a further object of the present invention to provide an all-suture knotless repair system wherein the bone anchor includes an anchor body having threads for attachment of the anchor body to bone.

It is also an object of the present invention to provide an all-suture knotless repair system wherein the bone anchor is an all-suture anchor assembly.

It is another object of the present invention to provide a method for securing tissue to bone using an all-suture knotless repair system. The method comprises first passing the plurality of sutures secured to the bone anchor through tissue. Thereafter, the second ends of the plurality of sutures are passed between the anchor suture strand and the suture anchor at positions between suture aperture locations defined by positions wherein the anchor suture strand has been passed through the suture anchor so as to penetrate and traverse the suture anchor in a manner defining apertures in the suture anchor. Finally, the all-suture anchor assembly is inserted into a bone hole.

It is further an object of the present invention to provide a method for securing tissue to bone using an all-suture knotless repair system wherein the step of inserting the all-suture anchor assembly includes inserting the suture anchor and a central portion of the anchor suture strand into the bone hole.

It is also an object of the present invention to provide a method for securing tissue to bone using an all-suture knotless repair system wherein the step of inserting the all-suture anchor assembly includes first inserting the anchor suture strand and the suture anchor into the bone hole in an elongated fashion to take a small diameter configuration.

It is another object of the present invention to provide a method for securing tissue to bone using an all-suture knotless repair system wherein the step of passing second ends of the plurality of sutures between the anchor suture strand and the suture anchor includes tensioning the second ends of the plurality of sutures.

It is further an object of the present invention to provide a method for securing tissue to bone using an all-suture knotless repair system wherein the step of inserting includes, after first inserting the anchor suture strand and the suture anchor, deploying the all-suture anchor assembly by tensioning the anchor suture strand to cause the suture anchor to fold, bend, crease, crinkle, bunch or otherwise change shape in a manner that compresses the suture anchor so as to increase the suture anchor in size in a direction substantially perpendicular to a longitudinal axis of the bone hole.

It is also an object of the present invention to provide a method for securing tissue to bone using an all-suture knotless repair system wherein the step of passing second ends of the plurality of sutures between the anchor suture strand and the suture anchor includes tensioning the second ends of the plurality of sutures.

It is another object of the present invention to provide a method for securing tissue to bone using an all-suture knotless repair system wherein the step of passing second ends of the plurality of sutures between the anchor suture strand and the suture anchor includes tensioning the second ends of the plurality of sutures.

It is further an object of the present invention to provide a method for securing tissue to bone using an all-suture knotless repair system wherein the bone anchor is an all-suture anchor assembly.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view showing the all-suture knotless repair system secured to tissue with the suture anchor in a relatively expanded configuration.

FIG. 2 is a perspective view showing the all-suture knotless repair system secured to tissue with the suture anchor in a relatively compressed configuration.

FIG. 3 is a detailed perspective view of the all-suture anchor assembly.

FIG. 4 is a cross sectional side view showing the all-suture knotless repair system being anchored within a bone hole.

FIG. 5 is a detailed perspective view of an alternate embodiment of the all-suture anchor assembly.

FIG. 6 is a cross sectional side view showing the alternate embodiment of the all-suture knotless repair system of FIG. 5 being anchored within bone.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

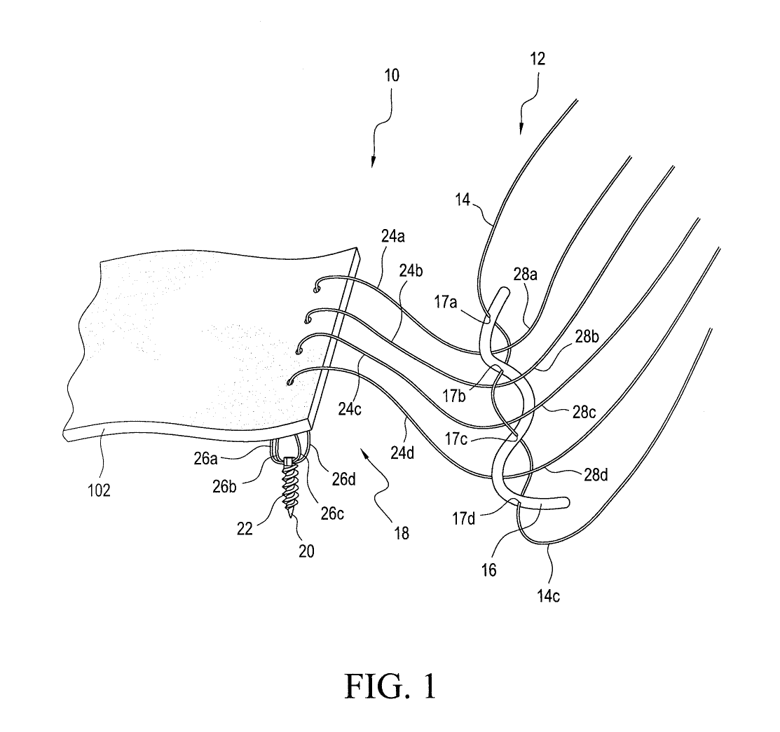

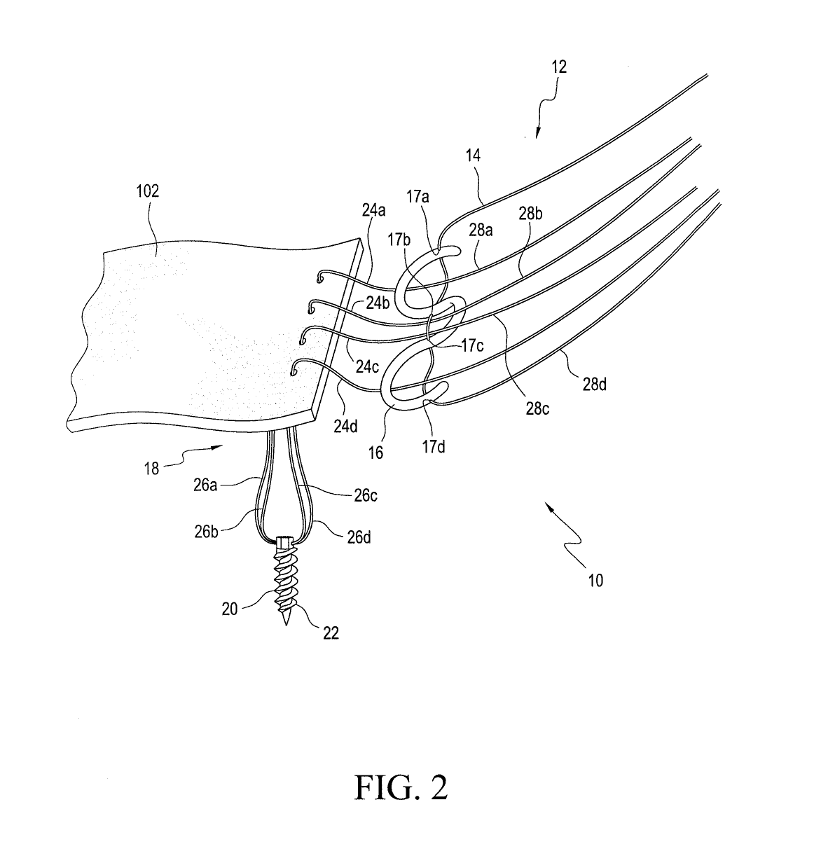

In accordance with the present invention, and with reference to FIGS. 1-4, an all-suture knotless repair system 10 is disclosed. As will be appreciated based upon the following disclosure, the all-suture knotless repair system 10 includes at least one all-suture anchor assembly 12 composed of an anchor suture strand 14 and a suture anchor 16 (composed entirely of suture material as will be discussed below in greater detail and therefore may be referred to as an all-suture anchor), a tissue coupling suture assembly 18 with a bone anchor 20, and a delivery inserter 30.

Considering first the all-suture anchor assembly 12, it is composed of an anchor suture strand 14 and a suture anchor 16 that are entangled in a manner facilitating bunching of the suture anchor causing compression of the suture anchor 16 in a manner creating outward forces that allow for placement and anchoring of the suture anchor 16 within a bone hole 100. In particular, the suture anchor 16 is preferably composed solely of an enlarged piece of cylindrical suture material or a suture tape. The enlarged surface area of the suture anchor 16 allows for the passage of the anchor suture strand 14 therethrough in a manner providing for entanglement of the suture anchor 16 and the anchor suture strand 14. The suture anchor 16 includes a first end 16a and a second end 16b, as well as a thickness, a width and a length along a longitudinal axis.

As briefly mentioned above, the all-suture anchor assembly 12 includes the anchor suture strand 14, which is passed through the suture anchor 16 at various locations 17a-d along the length of the suture anchor 16. That is, the anchor suture strand 14 penetrates and traverses the suture anchor 16 so as to define apertures in the suture anchor 16. The intersections of the anchor suture strand 14 with the suture anchor 16 are referred to herein as suture aperture locations and, as such, each of the suture aperture locations 17a-d is a location where the anchor suture strand 14 passes through the thickness of the suture anchor 16. As shown in FIGS. 1, 2 and 4, there are four suture aperture locations 17a-d. It is appreciated that as few as three suture aperture locations on a particular all-suture anchor assembly 12 may function well. Similarly, more suture aperture locations may be provided, although it has been discovered that each additional suture aperture location increases friction against the anchor suture strand 14 thus reducing a surgeon's ability to slide the anchor suture strand 14 in relation to the suture anchor 16. While the suture aperture locations 17a-d disclosed above in accordance with a preferred embodiment are all centrally located along the suture anchor 16 so as to be oriented along the central longitudinal axis of the suture anchor 16, it is appreciated the suture aperture locations may be varied (for example, staggered on opposite sides of the central longitudinal axis of the suture anchor) without departing from the spirit of the present invention. In accordance with a preferred embodiment, the suture anchor 16 may have various length and width dimensions depending upon the purpose for which it is intended.

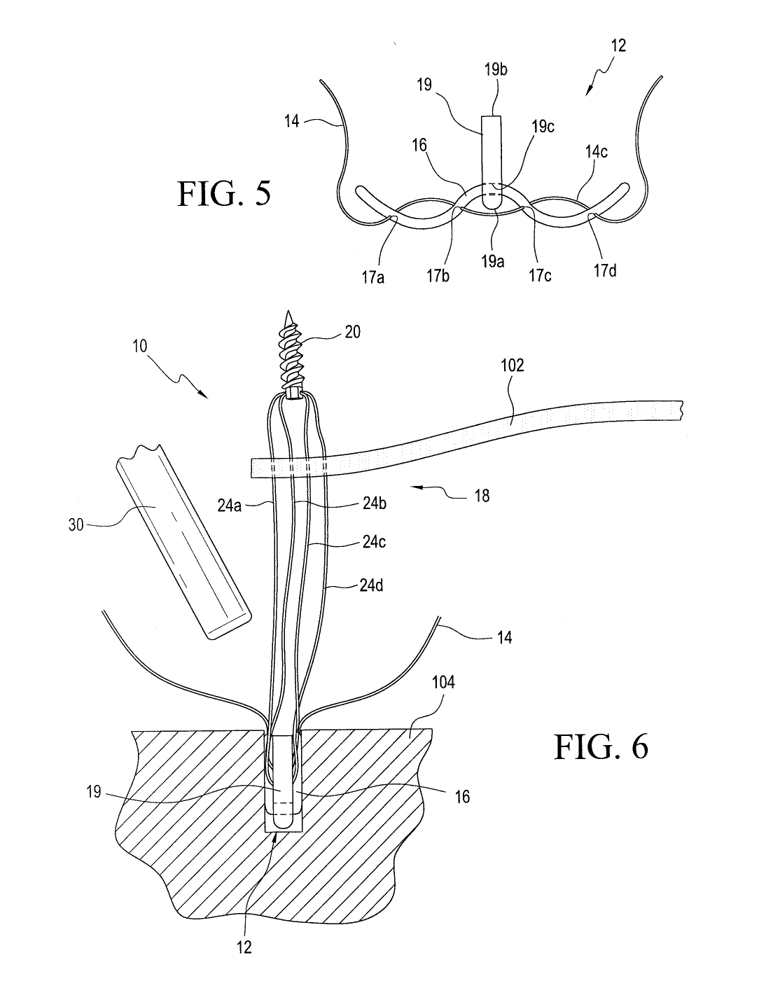

Referring to FIGS. 5 and 6, it is further contemplated the all-suture anchor assembly 12 may be supplemented with the inclusion of a solid polymer tip member 19 that is integrated onto the suture anchor 16 for the purpose of enhancing the surgeon's ability to position the all-suture anchor assembly 12 within the bone 104. In accordance with a preferred embodiment, the solid tip member 19 is cylindrical in shape and includes a forward first end 19a and a rear second end 19b. The forward first end 19a is semispherical in shape to enhance penetration as the tip member 19 is forced into bone 104 as discussed below. The tip member 19 is preferably secured to the suture anchor 16 by passing the suture anchor 16 through a lateral aperture 19c formed at the forward first end 19a of the tip member 19. Functionality in conjunction with the tip member 19 may be further enhanced by the provision of spikes or ridges along the outer surface thereof. Other than the inclusion of the tip member 19 along the length of the suture anchor 16, the structure, function, and operability of the all-suture anchor assembly 12 will remain the same.

As mentioned above, the all-suture knotless repair system 10 also includes a tissue coupling suture assembly 18 with a bone anchor 20. The tissue coupling suture assembly 18, therefore, includes the anchor 20 that has an anchor body 22. The anchor body 22 has traditional threads for attachment of the anchor body 22 to bone 104 in a manner known to those skilled in the art. With this in mind, it is appreciated any method of bone fixation can be used in conjunction with this anchor, including, but not limited to, wedge shaped, spikes, prongs, threads, or all-suture. While an embodiment employing a traditional anchor is disclosed, it is appreciated various anchoring systems may be employed. In fact, and as will be explained below in greater detail, the all-suture anchor assembly 12 described above may be employed as a replacement for the traditional bone anchor 20 described above and, as such, the all-suture anchor assembly 12 may be employed in securing the tissue coupling suture assembly 18 to bone 104.

A plurality of sutures 24a-d extend from and are secured to the anchor body 22, wherein each of the sutures 24a-d includes a first end 26a-d secured to the anchor body 22 and a second end 28a-d that is ultimately coupled with the all-suture anchor assembly 12. In this way, and as will be appreciated based upon the following disclosure, the present all-suture knotless repair system 10 is particularly well suited for extending the plurality of sutures 24a-d between anchored locations defined by the anchor 20 and the all-suture anchor assembly 12, with tissue 102 held therebetween, wherein the anchor body 22 fixedly secures the first ends 26a-d of the plurality of sutures 24a-d at one location and the all-suture anchor assembly 12 secures the second ends 28a-d of the plurality of sutures 24a-d at a second location. In this way, the present all-suture knotless repair system 10 is especially well adapted for creating a “dual-row” repair. However, it is also appreciated a variety of other uses and techniques may be implemented within the spirit of the present invention.

With regard to the attachment of the first ends 26a-d of the plurality of sutures 24a-d to the anchor body 22, the first ends 26a-d may be secured to the anchor body 22 in a fixed manner or in a sliding manner, both of which are well known to those skilled in the art. While the disclosed embodiment shows four lengths of suture, each having one end fixed to the anchor body, other configurations are contemplated. For example, one or more of the sutures may be slideable with respect to the anchor body. In some embodiments, the sutures and/or suture anchor may be configured to be moved relative to the anchor body and then fixed either by a knot or other technique. In other embodiments, the slideable suture may become fixed while the anchor is inserted into bone, such as by compressing the suture between the side of the anchor and the bone.

With regard to the attachment of the second ends 28a-d of the plurality of sutures 24a-d to the all-suture anchor assembly 12, the second ends 28a-d are passed between the anchor suture strand 14 and the suture anchor 16 at positions between the suture aperture locations 17a-d such that when the all-suture anchor assembly 12 is contracted to its compressed state, whether within a bone hole 100 or at another location within the body, friction is created between the second ends 28a-d of the plurality of sutures 24a-d and the all-suture anchor assembly 12 in a manner securing the second ends 28a-d of the plurality of sutures 24a-d to the all-suture anchor assembly 12.

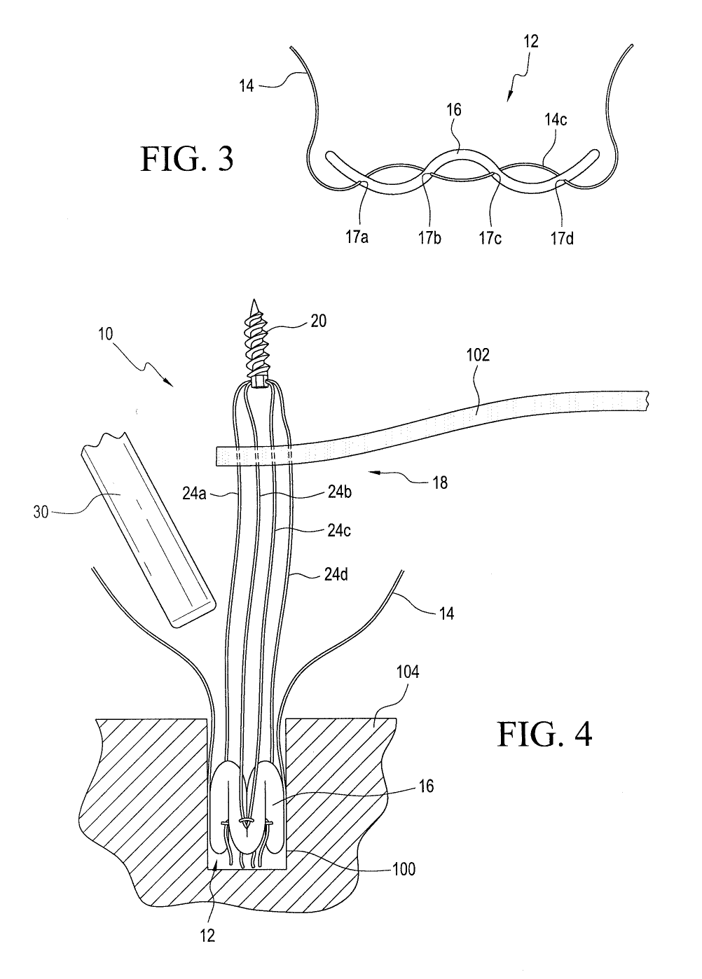

In particular, attachment of the all-suture anchor assembly 12 to bone 100 is achieved in the following manner. Referring now to FIG. 4, where the delivery inserter 30, the all-suture anchor assembly 12 and the tissue coupling suture assembly 18 (with a bone anchor 20) are shown, the plurality of sutures 24a-d are first passed through the tissue 102 to be attached. Thereafter, the second ends 28a-d of the plurality of sutures 24a-d are passed between the anchor suture strand 14 and the suture anchor 16 at positions between the suture aperture locations 17a-d. The all-suture anchor assembly 12, in particular, the suture anchor 16 and the central portion 14c of the anchor suture strand 14, are then inserted into the bone hole 100. It should be appreciated that the relative fit of the all-suture anchor assembly 12 in the bone hole 100 is shown as being relatively “loose.” This is done to provide for a clear view for the elements making up the present invention. In practice, it is appreciated that the anchor suture strand 14, suture anchor 16, and delivery inserter 30 would be tightly pressed into the bone hole 100, as any excess space would need to be taken up by the expansion of the all-suture anchor assembly 12 in a direction substantially perpendicular to the longitudinal axis of the bone hole 100 (or otherwise oriented to contact side walls of the bone hole 100).

As shown, the anchor suture strand 14 and the suture anchor 16 are installed in an elongated fashion to take a small diameter configuration, referred to herein as an undeployed state or installation state. Deployment preferably occurs as the delivery inserter 30 is removed and the anchor suture strand 14 is tensioned causing the suture anchor 16 to fold, bend, crease, crinkle, bunch or otherwise change shape in a manner that compresses the suture anchor 16 (however, it is appreciated deployment may occur before, after, or as the delivery inserter is removed). As the suture anchor 16 is compressed in this manner it ultimately increases in size in a direction substantially perpendicular to the longitudinal axis of the bone hole 100 (or otherwise oriented to contact side walls of the bone hole 100) and develops an outwardly directed forced that is directed at the walls of the bone hole 100. As such, and when tension is no longer being applied by the delivery inserter 30, or other delivery instrument, the suture anchor 16 exhibits an outward bias resulting in expansion of the suture anchor 16 in a direction substantially perpendicular to the longitudinal axis of the bone hole 100 (or otherwise oriented to contact side walls of the bone hole 100) into which it is positioned, resulting in frictional engagement or gripping the wall of the cancellous bone, which is referred to herein as the expanded deployed state.

It is appreciated a mechanical tensioning mechanism, as is well known to those skilled in the art, may be used during the deployment of the suture anchor 16. Such mechanical tensioning mechanisms pull or ratchet the suture strand 14 while the delivery inserter 30 holds the suture anchor 16 in place. Mechanical tensioning, of this nature, may be preferable as this can more tightly ‘fold’ or ‘bunch’ the all-suture anchor 16, thereby increasing the created tension, that is, the outward force of the suture anchor 16, which is relative to the longitudinal axis of the bone hole 100 and toward the walls of the bone hole 100, resulting from increased compression of the suture anchor 16. The changed shape of the suture anchor 16 provides security within the bone 104, for example, below the cortical layer.

Where the all-suture anchor assembly 12 including a solid tip member 19 as discussed above with reference to FIGS. 5 and 6 is employed, the methodology may be altered considering a bone hole may not be required, and the solid tip member 19 may be used to penetrate the bone 104 and form a cavity into which the all-suture anchor assembly 12 is positioned. In accordance with such an embodiment, the forward first end 19a of the solid tip member 19 is impacted directly into bone 104, with the remainder of the solid tip member 19 following and being inserted into the bone 104. Thereafter, the suture anchor 16 and the anchor suture strand 14 are forced into the hole created by the solid tip member 19, and the suture anchor 16 is folded, bent, creased, crinkled, bunched or otherwise changed in shape in a manner that compresses the suture anchor 16 as discussed above. Other than the inclusion of the solid tip member 19 and the direct impacting into the bone 104, the method disclosed herein remains the same.

It is also appreciated that a sleeve (likely threaded) might be inserted into the bone hole and the all-suture anchor is then placed into the sleeve. One goal of such a sleeve would be to enhance fixation of the all-suture anchor. In accordance with such an embodiment, the suture anchor would achieve fixation by gripping the walls of the sleeve. While a screw-in method of insertion is contemplated with the threaded construction, the sleeve may also be pressed into position or deployed in some other manner. The sleeve need not be uniform, so long as it can be secured within the bone hole, likely along the perimeter thereof. Alternatively, the suture anchor can change shape ‘distal’ to the sleeve thereby providing fixation. The sleeve would be positioned in the cortical bone aspect of the bone hole, but could also extend into the cancellous bone. It is, however, appreciated the sleeve could be slightly proud and not flush with the cortical surface, or it could be placed below the cortical surface, as long as it is well-fixed. It is also appreciated that when the suture anchor is deployed distal to the sleeve, that is, between the sleeve and the bottom of the bone hole, fixation doesn't require ‘force’. Rather, the enlargement and increased size (in a direction toward the walls of the bone hole) of the all-suture anchor upon deployment, distal to the sleeve after insertion, prevents the suture anchor from backing out of the sleeve (and ultimately from backing out of the bone hole).

Prior to expansion and folding (or otherwise changing the shape) of the anchor suture 16, the second ends 28a-d of the plurality of sutures 24a-d are tensioned as desired. With the sutures 24a-d tensioned, the anchor suture strand 14 is tensioned (either by manually or mechanically pulling this anchor suture strand 14) to cause the suture anchor 16 to fold and expand as explained above. In addition to causing the suture anchor 16 to grip the wall of the cancellous bone hole 100, the tensioning of the anchor suture strand 14 and the resulting folding of the suture anchor 16 causes the frictional engagement between the all-suture anchor assembly 12 and the second ends 28a-d of the plurality of sutures 24a-d. In particular, and as the suture anchor 16 is compressed during the deployment process, the suture anchor 16, the anchor suture strand 14 and the plurality of sutures 24a-d are brought closer together in a tightly compacted and entangled configuration that effectively creates a unitary mass that ultimately prevents the sutures 24a-d from being pulled away from the anchor suture 16.

As shown, the suture anchor 16 is folded or otherwise compressed to form pleats between adjacent suture aperture locations 17a-d. This pleating reduces the distance between the first suture aperture location 17a and the second suture aperture location 17b, as measured along the length of the suture anchor 16. These pleats form a bunched mass of suture material effectively increasing a diameter, or cross sectional dimension, (as measure in relation to the axis of the bone hole 100) of the suture anchor 16, which ultimately causes the all-suture anchor assembly 12 to displace cancellous bone. The relative increase in the cross sectional size of the suture anchor 16 in the direction substantially perpendicular to the longitudinal axis of the bone hole 100 (or in another direction to facilitate contact of the suture anchor 16 with the side walls of the bone hole 100) creates a retention force of the all-suture anchor assembly 12.

It is appreciated that increasing the number of suture aperture locations 17a-d from the four shown to five, six, seven or more is likely to increase the size of the all-suture anchor assembly 12 after deployment and the number of pleats is therefore increased. However, a limiting factor is the amount of friction increased by additional suture aperture location.

With the foregoing in mind, it is appreciated that a large variety of constructions and materials will work for the all-suture anchor assembly 12. It has been discovered that for each type of construction (i.e., braided, woven, non-woven, or knitted) there is an advantage for using a material that increases in width for every reduction in length. This advantage provided for increased diameters for a particular number of folds, pleats, crinkles or other changes in the shape of the suture anchor 16. Regardless of the material chosen for use in accordance with the present invention, the material must exhibit desirable deformation and retention characteristics.

As briefly mentioned above, it is contemplated the conventional anchor body 22 secured to the first ends 26a-d of the sutures 24a-d may be replaced with a second all-suture anchor assembly 12 as described above. In accordance with such an embodiment, the all-suture anchor assembly 12 of the present invention would be used in fixedly securing both the first and second ends 26a-d, 28a-d of the sutures 24a-d to the bone or other body part being secured.

The all-suture knotless repair system 10 described above may be employed in a variety of medical procedures. Included amongst those medical procedures that may take advantage of the present all-suture knotless repair system 10 are those procedures disclosed in U.S. Patent Application Publication No. 2015/0216522, entitled “SUTURE ANCHOR,” published Aug. 6, 2015, which is incorporated herein by reference. Briefly, and considering the procedures disclosed in the '522 publication, it is important to note that 1) one or more medial row anchors can be used; 2) one or more lateral row anchors can be used; and 3) all, or only some, of the sutures from one medial row anchor can be linked to one, or more than one, lateral row anchor (therefore, sutures form multiple medial row anchors may be crossed to multiple lateral row anchors).

While the preferred embodiments have been shown and described, it will be understood that there is no intent to limit the invention by such disclosure, but rather, is intended to cover all modifications and alternate constructions falling within the spirit and scope of the invention.

1. An all-suture knotless repair system, comprising:
at least one all-suture anchor assembly composed of an anchor suture strand and a suture anchor, the suture anchor consisting essentially of suture material;
a tissue coupling suture assembly with a bone anchor; and
a delivery inserter.

2. The all-suture knotless repair system according claim 1, wherein the anchor suture strand and the suture anchor are entangled in a manner facilitating bunching of the suture anchor causing compression of the suture anchor in a manner creating outward forces that allow for placement and anchoring of the suture anchor within a bone hole.

3. The all-suture knotless repair system according claim 2, wherein the suture material is an enlarged piece of cylindrical suture material or a suture tape.

4. The all-suture knotless repair system according claim 2, wherein the suture anchor includes a first end and a second end, and the anchor suture strand is passed through the suture anchor at suture aperture locations along a length of the suture anchor so as to penetrate and traverse the suture anchor in a manner defining apertures in the suture anchor, intersections of the anchor suture strand with the suture anchor are the suture aperture locations.

5. The all-suture knotless repair system according claim 4, wherein the bone anchor includes an anchor body having threads for attachment of the anchor body to bone.

6. The all-suture knotless repair system according claim 5, wherein a plurality of sutures extend from and are secured to the anchor body, each of the sutures includes a first end secured to the anchor body and a second end that is selectively coupled with the all-suture anchor assembly such that the anchor body fixedly secures the first ends of the plurality of sutures at one location and the all-suture anchor assembly secures the second ends of the plurality of sutures at a second location.

7. The all-suture knotless repair system according claim 6, wherein the second ends of the plurality of sutures are secured to the all-suture anchor assembly by passing the second ends of the plurality of sutures between the anchor suture strand and the suture anchor at positions between the suture aperture locations such that when the all-suture anchor assembly is contracted to its compressed state friction is created between the second ends of the plurality of sutures and the all-suture anchor assembly in a manner securing the second ends of the plurality of sutures to the all-suture anchor assembly.

8. The all-suture knotless repair system according claim 1, wherein the bone anchor includes an anchor body having threads for attachment of the anchor body to bone.

9. The all-suture knotless repair system according claim 8, wherein a plurality of sutures extend from and are secured to the anchor body, each of the sutures includes a first end secured to the anchor body and a second end that is selectively coupled with the all-suture anchor assembly such that the anchor body fixedly secures the first ends of the plurality of sutures at one location and the all-suture anchor assembly secures the second ends of the plurality of sutures at a second location.

10. The all-suture knotless repair system according claim 9, wherein the second ends of the plurality of sutures are secured to the all-suture anchor assembly by passing the second ends of the plurality of sutures between the anchor suture strand and the suture anchor at positions between the suture aperture locations such that when the all-suture anchor assembly is contracted to its compressed state friction is created between the second ends of the plurality of sutures and the all-suture anchor assembly in a manner securing the second ends of the plurality of sutures to the all-suture anchor assembly.

11. The all-suture knotless repair system according claim 1, wherein the bone anchor is an all-suture anchor assembly.

12. A method for securing tissue to bone using an all-suture knotless repair system, wherein all-suture knotless repair system includes an all-suture anchor assembly including at least one all-suture anchor assembly composed of an anchor suture strand and a suture anchor, the suture anchor consisting essentially of suture material, a tissue coupling suture assembly with a bone anchor having a plurality of sutures secured thereto, and a delivery inserter, the method comprising:
passing the plurality of sutures secured to the bone anchor through tissue;
passing second ends of the plurality of sutures between the anchor suture strand and the suture anchor at positions between suture aperture locations defined by positions wherein the anchor suture strand has been passed through the suture anchor so as to penetrate and traverse the suture anchor in a manner defining apertures in the suture anchor;
inserting the all-suture anchor assembly into a bone hole.

13. The method according to claim 12, wherein the step of inserting the all-suture anchor assembly includes inserting the suture anchor and a central portion of the anchor suture strand into the bone hole.

14. The method according to claim 13, wherein the step of inserting the all-suture anchor assembly includes first inserting the anchor suture strand and the suture anchor into the bone hole in an elongated fashion to take a small diameter configuration.

15. The method according to claim 14, wherein the step of passing second ends of the plurality of sutures between the anchor suture strand and the suture anchor includes tensioning the second ends of the plurality of sutures.

16. The method according to claim 14, wherein the step of inserting includes, after first inserting the anchor suture strand and the suture anchor, deploying the all-suture anchor assembly by tensioning the anchor suture strand to cause the suture anchor to fold, bend, crease, crinkle, bunch or otherwise change shape in a manner that compresses the suture anchor so as to increase the suture anchor in size in a direction substantially perpendicular to a longitudinal axis of the bone hole.

17. The method according to claim 16, wherein the step of passing second ends of the plurality of sutures between the anchor suture strand and the suture anchor includes tensioning the second ends of the plurality of sutures.

18. The method according to claim 12, wherein the step of passing second ends of the plurality of sutures between the anchor suture strand and the suture anchor includes tensioning the second ends of the plurality of sutures.

19. The method according to claim 12, wherein the bone anchor is an all-suture anchor assembly.
